SEQUENCE LISTING (3 sequences)

SEQ ID NO: 1 - DNA, 20 bp, Pseudomonas graminis
agagtttgatcctggctcag

SEQ ID NO: 2 - DNA, 19 bp, Pseudomonas graminis
ggttaccttgttacgactt

SEQ ID NO: 3 - DNA, 1402 bp, Pseudomonas graminis
accgtcctcccgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttctgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatccatctctggaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaaaagctcaaggcttccaacggctagttgacatcgtttacggcgtggactaccagggtatctaatcctgtttgctccccacgctttcgcacctcagtgtcagtatgagcccaggtggtcgccttcgccactggtgttccttcctatatctacgcatttcaccgctacacaggaaattccaccaccctctgccctactctagcttgccagttttggatgcagttcccaggttgagcccggggatttcacattcaacttaacaaaccacctacgcgcgctttacgcccagtaattccgattaacgcttgcaccctctgtattaccgcggctgctggcacagagttagccggtgcttattctgtcggtaacgtcaaaacagcaaggtattcgcttactgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgtccctttcgagacgttgtcccccactaccaggcagattcctaggcattactcacccgtccgccgctgaatcagagagcaagctctcttcatccgctcgacttgc

(19) United States
(12) Patent Application Publication
(10) Pub. No.: US 2023/0242868 A1
(43) Pub. Date: Aug. 3, 2023

(54) PSEUDOMONAS GRAMINIS STRAIN CAPABLE OF DEGRADING CELLULOSE AT LOW TEMPERATURE AND USE THEREOF

(71) Applicant: BEIJING ZNFY GROUP CO LTD., Beijing (CN)

(72) Inventor: Qingtao Qu, Beijing (CN)

(21) Appl. No.: 17/423,615

(22) PCT Filed: Aug. 26, 2020
(86) PCT No.: PCT/CN2020/111196

§ 371 Date: Jul. 16, 2021

(30) Foreign Application Priority Data
Dec. 30, 2019 (CN) 201911394095.9

(51) Int. Cl. C12N 1/20; C05F 11/08

(52) U.S. Cl. CPC C12N 1/205; C05F 11/08; C12R 2001/38

(57) ABSTRACT

A Pseudomonas graminis strain capable of degrading cellulose at a low temperature and use thereof. The accession number of the Pseudomonas graminis strain is CGMCC NO.: 18751. The strain has relatively strong capability of degrading cellulose at both a low temperature of 4° C. and a condition of 10° C., and the cellulose degradation capacities are basically the same. Although the cellulose degradation capacity is weakened at 30° C., the degradation amplitude is not large, and meanwhile, a KY240 strain also has potassium and inorganicphosphorus degradation capacity, and is expected to be used for preparing a bacterial fertilizer for degrading cellulose, promoting straw decomposition and improving soil fertility, particularly improving the amount of potassium and phosphorus available for plants in soil.

### TECHNICAL FIELD

The present disclosure relates to a Pseudomonas graminis strain capable of degrading cellulose at a low temperature, and use thereof.

### BACKGROUND

Lignocellulose is the most abundant and cheapest renewable resource on earth. The lignocellulose includes three kinds of polymers: cellulose, hemicellulose and lignin. The filaments of cellulose are closely intertwined and inter-embedded with lignin and hemicellulose in vascular tissues of cell walls of a higher plant through covalent bonds and non-covalent bonds, so as to form the lignocellulose. The quantity and kind of the lignocellulose will be different in different varieties of plants, different ages of plants and different parts of the same plant. Generally, the lignocellulose includes 39% of cellulose, 30% of hemicellulose, and 25% of lignin^{[1]}. Cellulose is a linear polymer with cellobiose (glucose-β-1,4-glucose) as the basic repeating unit and composed of thousands of glucose molecules. A β-1,4-linked glucose residue in a chain of cellulose rotates 1800 relative to an adjacent unit, the intrachain stability is maintained by hydrogen bonds, and the structure of cellulose has little change among species. Cellulose is a main component of a cell wall of a plant, accounting for more than 90% of the total amount of biomass resources. It is the most abundant biomass resource in nature. Therefore, the degradation of cellulose is a breakthrough point in the utilization of lignocellulose resources. It is estimated that the lignocellulose accounts for more than 60% of photosynthesis products on earth, and the annual production of cellulose in the world is as high as 100 billion tons^{[2]}. The use of abundant and cheap cellulose materials will be one of the ways for people to solve the future food and energy problems, which has far-reaching significance.

However, except a small part of natural fiber substances can be utilized inefficiently by ruminants, most of the natural fiber substances are burned in situ or exist in the natural environment in a waste form, which pollutes the environment while wasting energy^{[3-4]}. Currently, for the utilization of cellulose, recycling is mainly realized by chemical or biological treatment. Cellulose has an insoluble rigid structure, and is insoluble in water, dilute acid and dilute alkali at normal temperature^{[5]}. It decomposes slowly under natural conditions, and the insolubility and heterogeneity of cellulose also make it difficult to degrade cellulose^{[6]}, so it is difficult to be used as an industrial raw material and feed. Therefore, microbial decomposition of cellulose has become the core of cellulose biological treatment technology. In order to make full use of this kind of abundant renewable resources, scholars began to study the biodegradation of cellulose as early as 1883 and 1886^{[7]}, but the research on the real use of microorganisms to degrade cellulose began in the 1960s, at which time microorganisms were mainly used for degrading cellulose to produce single-cell proteins; and after 1970s, with the energy crisis and environmental pollution, the research focus gradually shifted to developing new energy sources and preventing environmental pollution^{[8]}.

Currently, most of research reports on cellulose at home and abroad are the research and development of microorganisms that degrade cellulose at medium and high temperatures, while the research on microorganisms that degrade cellulose at a low temperature is relatively few^{[9]}. The isolated microorganisms that produce low-temperature cellulase are also mainly concentrated in some marine fungilo^{[10-11]} and bacteria^{[12-14]} from the marine environment. However, degrading of cellulose by the bacteria at medium and high temperatures can only be carried out under certain high temperature conditions, which has a certain limitation on the temperature, and thus also wastes high-temperature energy sources. Therefore, low-temperature cellulose-degrading bacteria have certain advantages and great development prospects.

China is a large agricultural country with the highest sown area of crops in the world, and is also one of the countries with the most abundant straw resources in the world. The total number of crop straws produced every year is 687 million tons^{[15]}, of which 220 million tons are corn straws, which is a kind of biomass resource that urgently needs to be further developed and utilized^{[16]}. Northeast and northern areas are important grain reserve areas in China, and are areas with the largest reserves of plant lignocellulose. After harvest of crops, there will be a large number of crop straws waiting to be treated. Most of traditional treatment methods are burning and returning to field of straws. Due to the severe haze climate in the northern area, the burning treatment of straw will aggravate the air pollution problem. Therefore, the government has made control over burning of straws, and a large number of straws can only be decomposed by returning to the field. However, because the environment and temperature will directly affect the decomposition rate of straws, the cold conditions in Northeast and northern areas make it impossible to treat and utilize a large number of cellulose resources in time and effectively. Scholars have found through research that, the degradation rate of straws by microorganisms in soil is very slow when the soil temperature is lower than 10° C.; the degradation rate of straws by the microorganisms is the fastest when the temperature is in the range of 20° C.-30° C.; and the temperature is positively correlated with the degradation rate of straws when the soil temperature is lower than 30° C., and the degradation rate is slower when the temperature is lower^{[17]}. In most regions of northern China, in autumn and winter the temperature is low, the ice age is long, the climate is dry and cold, and the decomposition of straws after returning to field is slow, which leads to the failure of timely decomposition of straws after returning to field. A large number of straw residues remained in the soil directly affect the soil preparation of farmland and the sowing of crops in the next year, and meanwhile, it also causes some diseases and pests to survive in the soil for a long time, which will bring serious harm to crop growth. In order to ensure the normal sowing and growth of the crops, many places still adopt field burning to solve the influence of slow decomposition of straws after returning to field on the farmland and the crops. Although this temporarily solves the sowing problem of the crops, it also causes serious pollution to the atmospheric environment at the same time, so it is still impossible to solve the problem of treating and utilizing a large number of straws reasonably and effectively. Therefore, the research on screening strains that degrade cellulose at a low temperature has increasingly become a hot spot in research for domestic scholars. If the screened strains that degrade cellulose at a low temperature are utilized, the decomposition speed of the straws returned to field in a low-temperature environment can be accelerated, and thus the nutrients of the straws are fully decomposed and released, and transformed into simple organic matters that can be extremely easily absorbed and utilized by plants, thereby improving the utilization rate of the straws and thus increasing the yield. It will have an important application value.

Currently, there are few reports about low-temperature cellulase-producing bacteria. Mu Chunlei et al.^{[18]} isolated a fungus M11 which could decompose cellulose efficiently at 13° C. from the soil of the field to which the straws were returned, and identified MI1 as Penicillium oxalicum. Zhang Dan et al.^{[19]} isolated two bacterial strains B9 (Cytophaga, also known as Cytophage) and B21 (Cellulomonas), and determined that they could degrade cellulose obviously at 10-15° C. Meng Jianyu et al.^{[20]} isolated a low-temperature cellulase-producing strain with great development potential at 10° C. Zheng Guoxiang et al.^{[21]} isolated a strain L-11, which was identified as Penicillium olsonii, and it was determined that it had the function of degrading microcrystalline cellulose at a low temperature condition of 15° C. Zhao Xu et al.^{[22]} screened out a fungal strain D5 which could degrade carboxymethyl cellulose, corn straw cellulose and produce cellulase with high yield at 15° C. by utilizing 30 samples collected from mountainous regions of Weiyuan County, and the fungal strain was preliminarily identified as Penicillium sp. by ITS rDNA sequence analysis.

In view of the above, at the present stage, the research on bacteria that degrade cellulose at a low temperature mostly focuses on 10° C.-20° C., which brings certain advantages, but its application scope is still limited. Therefore, it is of very great practical and theoretical significance to screen out a functional microbial strain which can grow fast and degrade cellulose efficiently at a lower temperature (4° C.) and also can degrade cellulose effectively at a higher temperature.

Pseudomonas graminis is a novel strain discovered by Behrendt U, Ulrich A, Schumann P, et al.^{[23]}. Like other Pseudomonas, it is Gram-negative, aerobic and rod-shaped, and has bipolar flagellation. The isolate is catalase positive and oxidase negative, and cannot oxidize or ferment glucose by producing acid. The isolate do not reduce nitrate to nitrite, but can use various compounds, preferably a monosaccharide, independently as the only carbon source. The tested disaccharide is not used as a substrate. There is no data showing that it can degrade cellulose.

### CITED REFERENCES

[1] Freitag M, Morrell J J. Changes in selected enzyme activities during growth of pure and mixed cultures of the white-rot decay fungus Trametes versicolor and the potential biocontrol fungus Trichoderma harzianum[J]. Canadian Journal of Microbiology, 1992, 38(4):317-323.
[2] Wyman C E, Dale B E, Elander R T, et al. Coordinated Development of Leading Biomass Pretreatment Technolo-gies[J]. Bioresource Technology, 2005, 96(18):1959-1966.
[3] Lynd L R, Weimer P J, Willem H Z, et al. Microbial cellu-lose utilization: fundamentals and biotechnology[J]. Mol Biol Rew, 2002, 66:506-577.
[4] Souichiro K, Shin H. Effective cellulose degradation by a mixed-culture system composed of a cellulolytic Clostridi-um and aerobic non-cellulolytic bacteria[J]. FEMS Microbiology Ecology, 2004, 51:133-142.
[5] Chen Hongzhang. Cellulose Biotechnology [M]. Beijing: Chemical Industry Press, 2005:10-51.
[6] Luo Hui, Qiu Tianlei, Cheng Lei. Research progress of anaerobic degradation of cellulose [J]. China Biogas, 2008, 26(2):3-9.
[7] Wang Chunhui, Wang Tianhong. Advances on cellulose binding domain of microbiological cellulase [J]. Journal of Cellulose Science and Technology, 1997, 5(004):1-10.
[8] Wen Shaobai, Li Qinfen, Hou Xianwen. Recent Advances in Microbial Degradation of Cellulose [J]. Chinese Agricultural Science Bulletin 2010, 26(01):231-236.
[9] Singh R, Shukla A, Tiwari S, et al. A review on delignification of lignocellulosic biomass for enhancement of ethanol production potential [J]. Renewable and Sustainable Energy Reviews, 2014, 32:713-728.
[10] Chen Liang, Chi Naiyu, Zhang Qingfang. Breeding and Fermentation Medium Optimization of-Cold-active Cellulase Strain CNY086 (I) [j]. Microbiology China, 2009, 36 (10):1547-1552.
[11] Hou Yunhua. Molecular research on the cellulase and hemicellulase of a novel cold-adaptive marine Pencillium [D]. Jinan: Shandong University, 2006.
[12] Zeng Yin-xin, Yu Yong, Chen Bo, Li Huirong. Screening, classification and growth of bacterium producing cold-active cellulase and its enzymic characterization [J]. High Technology Letters, 2005, 15 (4):58-62.
[13] You yinwei, Wang Tianhong. Cloning and expression of gene of endoglucanase of marine cold-adaptive bacteria Pseudoalteromonas sp. MB-1 [J]. Microbiology China, 2005, 45 (1):142-144.
[14] Dang H Y, Zhu H, Wang J, Li T G. Extracellular hydrolytic enzyme screening of culturable heterotrophic bacteria from deep-sea sediments of the southern Okinawa Trough [J]. World J Microbiol Biotechnol, 2009, 25 (1): 71-79.
[15] Edited by the National Bureau of Statistics of the People's Republic of China. China Statistical Yearbook [M]. Beijing: China Statistics Press, 2010.
[16] Han Jiahui, Yang, Zhang Jinglai. Comparison of straw utilization patterns by regression model [J]. J Anhui Sci Technol Univ, 2009, 23(6):87-91.
[17] Zhang Hongyuan, Liu Mingzhong, Zhang Jiajian. Study on the decomposition law of organic materials in dryland soil [J]. Soils and Fertilizers, 1986, 4: 7-11.
[18] Mu Chunlei, Wu Xiaosen, Li Shuna. Screening and identification of a cold-adapted cellulase-producing strains and characterization of cellulase [J]. Microbiology China, 2013, 40(7):1193-1201.
[19] Zhang Dan, Xu Jinggang, Lu Weiming. Selection and identification of bacteria degrading textile fibers under low temperature [J]. Journal of Northeast Agricultural University, 2008, 39(1):55-57.
[20] Meng Jianyu, Li Heng, Fan Zhaoyang. Isolation and identification of cellulose-degrading bacteria under low temperature [J]. Chinese Journal of Applied and Environmental Biology, 2014, 20(1):152-156.
[21] Zheng Guoxiang, Ai Shuang, Yin Ting. Screen, identification and optimization of enzyme-producing conditions of a low temperature-resistant cellulose degradation strain [J]. Journal of Northeast Agricultural University, 201950(2):79-89.
[22] Zhao Xu, Wang Wenli, Li Juan. Isolation and Identification of Low-temperature Degrading Strains for Corn Stubble [J]. Soil and Crop, 2017, 6 (3):192-198.
[23] Behrendt U, Ulrich A, Schumann P, et al. A taxonomic study of bacteria isolated from grasses: a proposed new species Pseudomonas graminis sp. nov [J]. International Journal of Systematic and Evolutionary Microbiology, 1999, 49(1): 297-308.

### SUMMARY

An objective of the present disclosure is to provide a novel strain of Pseudomonas graminis, which can degrade cellulose at a low temperature (4° C.), and meanwhile also has the function of degrading potassium and inorganicphosphorus.

The technical solutions adopted by the present disclosure are as follows.

A strain KY240 is screened out from nearly 300,000 microbial strains by the inventor through massive screening on soil derived from all parts of China. The sequencing result of 16sDNA of this strain has showed that the strain is highly homologous to Pseudomonas graminis, with the homology of more than 99.97%. Furthermore, the research results also have showed that the strain also has the function of degrading potassium and inorganicphosphorus. It is the first time in the world that it is reported KY240 has the capability of degrading cellulose, potassium and inorganicphosphorus at a low temperature of 4° C., and has a promotion effect on the decomposition of wheat straws.

In a first aspect of the present disclosure, provided is:

a Pseudomonas graminis strain preserved in China General Microbiological Culture Collection Center (CGMCC) of Institute of Microbiology, Chinese Academy of Sciences, located at No. 3, No. 1 Courtyard, Beichen West Road, Chaoyang District, Beijing, with the accession number of CGMCC No.: 18751. The preservation date was Oct. 28, 2019, and the strain was registered in the Collection Center on the same day, and it was detected to determine whether the strain was survived. The suggested classification name of the strain was: Pseudomonas graminis.

In a second aspect of the present disclosure, provided is:

a microbial preparation including the Pseudomonas graminis strain according to the first aspect of the present disclosure.

In some examples of the microbial preparation, the microbial preparation is used in at least one of the following aspects:

degrading an organic matter containing cellulose;

promoting straw decomposition;

degrading potassium and inorganicphosphorus; and

preparing microbial fertilizer.

In a third aspect of the present invention, provided is:

use of the Pseudomonas graminis strain according to the first aspect of the present disclosure, including:

degrading an organic matter containing cellulose;

promoting straw decomposition;

degrading potassium and inorganicphosphorus; or

preparing microbial fertilizer.

In a fourth aspect of the present invention, provided is:

a method for promoting cellulose degradation, including culturing a mixture of the Pseudomonas graminis strain according to the first aspect of the present disclosure and cellulose.

In some examples of promoting cellulose degradation, the cellulose is derived from crop straws, feces of livestock, branches and leaves and residues of various plants, spent mushroom compost, residues after sugar making, dregs of a decoction of Chinese herbal medicines, wastes of beverage raw materials, and plant residues in farmland.

In some examples of promoting cellulose degradation, the culture temperature is 0-40° C.

In a fifth aspect of the present disclosure, provided is:

a method for degrading potassium, including culturing a mixture of the Pseudomonas graminis strain according to the first aspect of the present disclosure and a potassium-containing mineral.

In some examples of degrading potassium, the potassium-containing mineral is selected from potassium feldspar, plagioclase, microcline, illite, vermiculite, montmorillonite, silicate and mica.

In some examples of degrading potassium, mica includes biotite, phlogopite, muscovite and lepidolite.

In a sixth aspect of the present disclosure, provided is:

a method for degrading inorganicphosphorus, including culturing a mixture of the Pseudomonas graminis strain according to the first aspect of the present disclosure and a phosphorus-containing mineral.

In some examples of degrading inorganicphosphorus, the phosphorus-containing mineral is selected from various phosphorus-containing minerals such as tricalcium phosphate, rock phosphate powder, fluorapatite, chloroapatite, hydroxyapatite, goyazite, phosphate (vivianite), lignite, peat soil, wavellite, variscite and strengite.

The beneficial effects of the present disclosure are as follows.

The KY240 strain of the present disclosure can degrade cellulose at a low temperature of 4° C., and upon sequencing analysis of the 16sDNA of the strain, the strain is highly Y homologous to Pseudomonas graminis. Compared with a commercial strain Bacillus subtilis 92068, the KY240 strain has stronger capability of degrading cellulose at a low temperature of 4° C. and a condition of 10° C., and the cellulose degradation capacities are basically the same. Although the cellulose degradation capacity is weakened at 30° C., the degradation amplitude is not large, and meanwhile, the KY240 strain also has potassium and inorganicphosphorus degradation capacity, which has a promotion effect on the decomposition of wheat straws, and the effect is obviously superior to that of the commercial strain 92068. Therefore, KY240 is a functional microbial strain with both a wide working range and multiple functions, which can both degrade cellulose at a low temperature (4° C.) and medium-high temperatures (10° C.-30° C.), and has the capability of degrading both potassium and inorganicphosphorus, and has a promotion effect on the decomposition of wheat straws. It is expected to be used for preparing bacterial fertilizer for degrading cellulose, promoting straw decomposition, and improving soil fertility, especially increasing the amount of potassium and phosphorus in soil available to plants.

### BRIEF DESCRIPTION OF THE DRAWINGS

FIG. 1 shows the homologous comparison analysis (Neighbor-Join) of the KY240 strain and Pseudomonas graminis.

FIG. 2 shows the morphology of the KY240 strain under a microscope (10×100);

FIG. 3 shows the result of degradation of cellulose by the KY240 strain at 4° C.;

FIG. 4 shows the result of degradation of cellulose by the KY240 strain at 10° C.;

FIG. 5 shows the result of degradation of cellulose by the KY240 strain at 30° C.;

FIG. 6 shows the determination result of the potassium degrading capability of the KY240 strain;

FIG. 7 shows the determination result of the inorganicphosphorus degrading capability of the KY240 strain;

FIG. 8 shows the determination result of living bacteria count of the strains 92068 and KY240 at 30° C.;

FIG. 9 shows the determination result of living bacteria count of the strains 92068 and KY240 at 4° C.;

FIG. 10 shows the decomposition of wheat straws by the KY240 strain and the Bacillus subtilis 92068 strain at a low temperature of 4° C.

### DESCRIPTION OF THE EMBODIMENTS

After massive screening on soil from all parts of China, the inventor have screened out a strain from nearly 300,000 microbial strains, the strain can grow stably and rapidly and degrade cellulose stably at a low temperature of 4° C., and can also degrade cellulose at medium-high temperatures, and the strain is named as KY240. Furthermore, the strain has multiple functions of degrading mineral potassium and inorganicphosphorus.

Determination of 16sDNA and Physiological Morphology Analysis of KY240 Strain:
Determination of 16sDNA of Strain

### Extraction of Bacterial DNA by CTAB Method

1) A single colony was inoculated in 5 mL R2A, and cultured at 30° C. overnight.

2) 1 mL of a seed culture solution was inoculated into 100 ml of a R2A solution, and cultured at 37° C. and 220 r/min for 16 hours.

3) The culture was centrifuged at 5,000 r/min for 10 minutes, and the supernatant was discarded.

4) The precipitate was added with 10 mL TE for centrifugal washing, and then the bacteria were dissolved with 10 mL TE, mixed well, and stored at −20° C. for later use.

5) 3.5 mL of the bacterial suspension was taken, added with 184 μL of 10% SDS, mixed well, added with 37 μL of 10 mg/mL proteinase K, mixed well, and incubated at 37° C. for 1 hour.

6) It was added with 740 μL of 5 mol/L NaCl, then added with 512 μL of CTAB/NaCl, mixed well, and incubated at 65° C. for 10 min.

7) It was added with equal volume of chloroform/isoamylol, mixed well, and centrifuged at 10,000 r/min for 5 minutes, and the supernatant was retained.

8) The supernatant was added with equal volume of phenol: chloroform: isoamylol (25: 24: 1), mixed well, and centrifuged at 10,000 r/min for 5 minutes, and the supernatant was retained.

9) The supernatant was added with 0.6 times the volume of isopropanol, mixed well, and centrifuged at 10,000 r/min for 5 minutes, the DNA precipitate was collected and centrifuged, and the DNA precipitate was subjected to centrifugal washing with 70% ethanol.

10) The DNA was dissolved with 1 mL TE, added with RNaseA at a final concentration of 20 μg/mL, and stored at 4° C.

### Amplification and Sequencing

PCR amplification of 16S rDNA was conducted with 16S rDNA universal primers 27f (5′-AGAGTTTGATCCTGGCTCAG-3′ (SEQ ID NO.: 1)) and 1492r (5′-GGTTACCTTGTTACGACTT-3′ (SEQ ID NO.: 2)). PCR reaction conditions: pre-denaturation at 94° C. for 30 s; denaturation at 94° C. for 30 s, annealing at 52° C. for 30 s and extending at 72° C. for 60 s, and repeating these for 35 cycles. The PCR products were subjected to 1.5% agarose gel electrophoresis, which was followed by recovery, purification and sequencing (by Beijing meiyimei biotechnology Co., Ltd.). According to the obtained 16S rDNA sequence, the homologous sequences were searched by Blast in GenBank, and subjected to homologous sequence analysis and comparison, so as to establish a phylogenetic tree.

16S sequencing result of KY240 strain

The 16sDNA sequencing result of the KY240 strain was:

(SEQ ID NO.: 3)
ACCGTCCTCCCGAAGGTTAGACTAGCTACTTCTGGTGCAACCCACTCCCA
TGGTGTGACGGGCGGTGTGTACAAGGCCCGGGAACGTATTCACCGCGACA
TTCTGATTCGCGATTACTAGCGATTCCGACTTCACGCAGTCGAGTTGCAG
ACTGCGATCCGGACTACGATCGGTTTTCTGGGATTAGCTCCACCTCGCGG
CTTGGCAACCCTCTGTACCGACCATTGTAGCACGTGTGTAGCCCAGGCCG
TAAGGGCCATGATGACTTGACGTCATCCCCACCTTCCTCCGGTTTGTCAC
CGGCAGTCTCCTTAGAGTGCCCACCATAACGTGCTGGTAACTAAGGACAA
GGGTTGCGCTCGTTACGGGACTTAACCCAACATCTCACGACACGAGCTGA
CGACAGCCATGCAGCACCTGTCTCAATGTTCCCGAAGGCACCAATCCATC
TCTGGAAAGTTCATTGGATGTCAAGGCCTGGTAAGGTTCTTCGCGTTGCT
TCGAATTAAACCACATGCTCCACCGCTTGTGCGGGCCCCCGTCAATTCAT
TTGAGTTTTAACCTTGCGGCCGTACTCCCCAGGCGGTCAACTTAATGCGT
TAGCTGCGCCACTAAAAGCTCAAGGCTTCCAACGGCTAGTTGACATCGTT
TACGGCGTGGACTACCAGGGTATCTAATCCTGTTTGCTCCCCACGCTTTC
GCACCTCAGTGTCAGTATGAGCCCAGGTGGTCGCCTTCGCCACTGGTGTT
CCTTCCTATATCTACGCATTTCACCGCTACACAGGAAATTCCACCACCCT
CTGCCCTACTCTAGCTTGCCAGTTTTGGATGCAGTTCCCAGGTTGAGCCC
GGGGATTTCACATTCAACTTAACAAACCACCTACGCGCGCTTTACGCCCA
GTAATTCCGATTAACGCTTGCACCCTCTGTATTACCGCGGCTGCTGGCAC
AGAGTTAGCCGGTGCTTATTCTGTCGGTAACGTCAAAACAGCAAGGTATT
CGCTTACTGCCCTTCCTCCCAACTTAAAGTGCTTTACAATCCGAAGACCT
TCTTCACACACGCGGCATGGCTGGATCAGGCTTTCGCCCATTGTCCAATA
TTCCCCACTGCTGCCTCCCGTAGGAGTCTGGACCGTGTCTCAGTTCCAGT
GTGACTGATCATCCTCTCAGACCAGTTACGGATCGTCGCCTTGGTGAGCC
ATTACCTCACCAACTAGCTAATCCGACCTAGGCTCATCTGATAGCGCAAG
GCCCGAAGGTCCCCTGCTTTCTCCCGTAGGACGTATGCGGTATTAGCGTC
CCTTTCGAGACGTTGTCCCCCACTACCAGGCAGATTCCTAGGCATTACTC
ACCCGTCCGCCGCTGAATCAGAGAGCAAGCTCTCTTCATCCGCTCGACTT
GC.

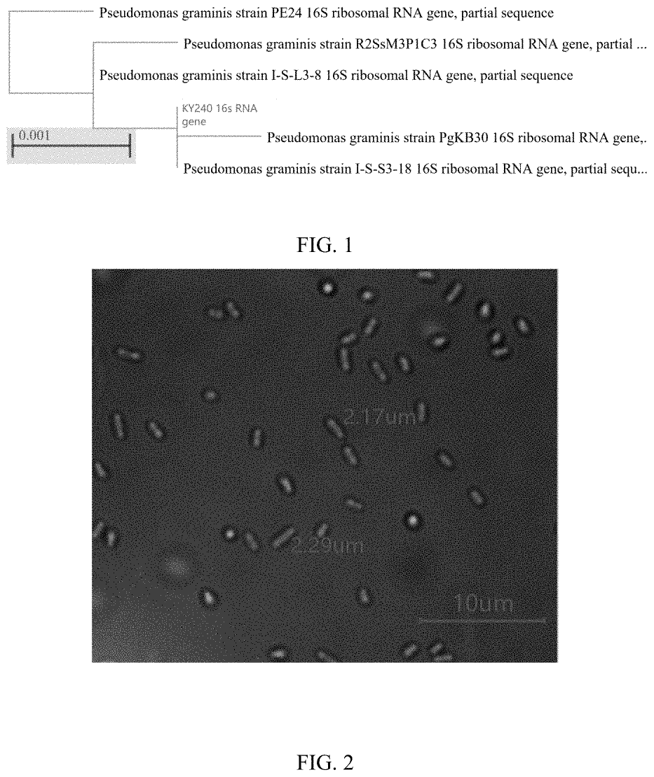

The sequencing result showed that the KY240 strain had a high degree of homology greater than 99.97% with Pseudomonas graminis (FIG. 1 was comparative analysis of homology between the KY240 strain and Pseudomonas graminis).

### Morphological Observation of KY240 Strain

### Morphological Observation of Strain

The screened strain was inoculated onto a R2A plate and cultured at 30° C. for 2 d. The size, shape, color, gloss, viscosity, bulge shape, transparency, edge characteristics and presence or absence of spores of the colony were observed.

### Morphology Observation Results of Strain

It was observed that when the KY240 strain (Pseudomonas graminis, Pseudomonas) was cultured and grown on the R2A medium for 2 d, the colony had a round shape, was yellow-green and opaque, had a smooth and moist surface, a regular edge, a halo and a convex center, and upon determination with a microscope, it had a diameter of about 2-3 μm, was round, shiny, sticky, slightly raised and had smooth and neat edges (FIG. 2, the scale length in the figure was 10 μm)

### Determination of Cellulose Degrading Capability of KY240 Strain

### Determination of Cellulose Degrading Capability of KY240 Strain at Different Temperatures

The KY240 strain, which had been cultured on the R2A medium for 2 d, was inoculated into a CMC medium, and Bacillus subtilis (the strain 92068) was set as a positive control. The KY240 strain and the 92068 strain were simultaneously and respectively cultured at 4° C. and 10° C. for 7 d, and cultured at 0° C. for 2 days, and then they were fumigated with an iodine solution, and determined for the diameter of a CMC degradation halo in mm.

CMC degrading capability=the diameter of the CMC degradation halo in mm+X

Note: X was a weighting coefficient, which was −1, 0, 1 and 2 accordingly according to the transparency of the degradation halo of the strain. The number 2 represented that the degradation halo was completely transparent; the number 1 represented that the degradation halo was subtransparent; and 0 represented that the degradation halo was opaque. However, there were traces of hydrolysis on the surface of the culture medium, and thus the degradation halo was basically cannot be observed by human eyes, but after the colony was washed with water, there are weak traces of hydrolysis at the place where the bacteria were inoculated, and −1 represented that there was no any hydrolysis activity. This method was also applied to test the potassium degrading capability and inorganicphosphorus degrading capability of bacteria. Determination results of cellulose degrading capability of KY240 strain

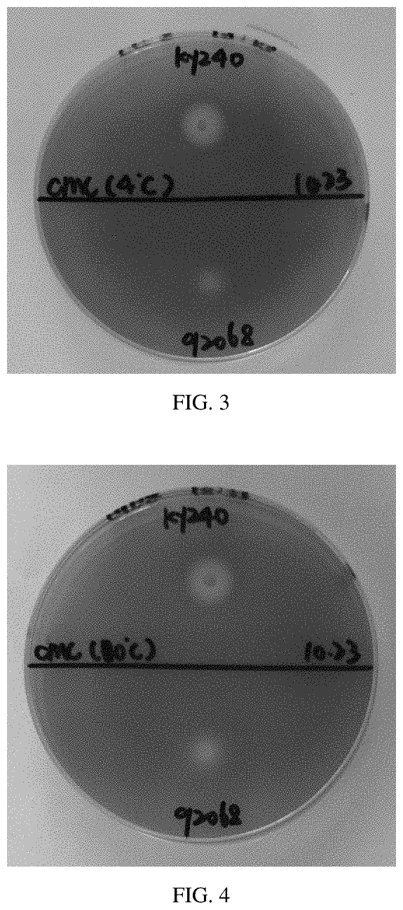

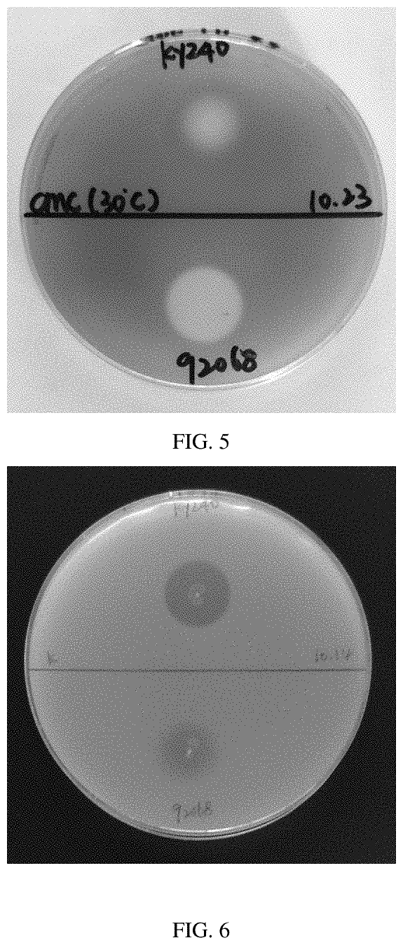

The determination results were as shown in Table 1 and FIGS. 3-5. The results showed that the cellulose degrading capability of the KY240 strain was obviously higher than that of the control Bacillus subtilis strain 92068 at 4° C. and 10° C.

TABLE 1
Cellulose degrading capability of strains on the
CMC medium at different temperatures

| | Cellulose degrading capability | |
|---|---|---|
| Culture | 92068 (CK) | KY240 |
| temperature | Bacillus subtilis | Pseudomonas graminis |
| 4° C. | <5 | 32.2 |
| 10° C. | <5 | 31.5 |
| 30° C. | 39.6 | 22.8 |

In view of the above, Bacillus subtilis (92068) could only degrade cellulose efficiently at 30° C. Compared with Bacillus subtilis (92068), KY240 had a strong cellulose degrading capability both at a low temperature of 4° C. and a condition of 10° C., and the cellulose degrading capabilities of it were basically the same. Although the cellulose degrading capability of it was decreased at the condition of 30° C., the decrease amplitude was not large. It could be seen from this that KY240 was a microbial strain with a wide working range, which could degrade cellulose at a low temperature (4° C.-10° C.) and could also degrade cellulose stably and efficiently at 30° C.

### Determination of Multifunctionality of KY240 Strain

### Determination of Potassium Degrading Capability of KY240 Strain

The KY240 strain which had been cultured on the R2A medium for 2 days, was inoculated into a potassium medium (10.0 g of glucose, 0.5 g of yeast powder, 1.0 g of ammonium sulfate, 2.0 g of disodium hydrogen phosphate, 0.5 g of magnesium sulfate heptahydrate, 1.0 g of calcium carbonate, 15.0 g of agar powder, 1.0 g of potassium feldspar, and 1,000 mL of water), and Bacillus subtilis (strain 92068) was set as a positive control. They were cultured at room temperature, the colonies were washed off with water, and the diameters of potassium degradation halos were determined in mm. potassium degrading capability=the diameter of the potassium degradation halo in mm+X

Note: X was a weighting coefficient, which was −2, −1, 0, 1 and 2 accordingly according to the transparency of the potassium degradation halo of the strain.

### Determination Results of the Potassium Degrading Capability of KY240 Strain

The results were as shown in Table 2 and FIG. 6. The results showed that the potassium degrading capability of the KY240 strain was obviously higher than that of the control Bacillus subtilis strain 92068.

TABLE 2
Potassium degrading capability of strains on a potassium medium

| | 92068 (CK) | KY240 |
|---|---|---|
| Strain | Bacillus subtilis | Pseudomonas graminis |
| Capability of | 10.7 | 36.2 |
| degrading | | |
| potassium | | |

### Determination of Inorganicphosphorus Degrading Capability of KY240 Strain

The KY240 strain which had been cultured on the R2A medium for 2 days, was inoculated into a inorganicphosphorus medium (10.0 g of glucose, 0.5 g of yeast extract, 0.5 g of ammonium sulfate, 0.02 g of potassium chloride, 0.02 g of sodium chloride, 0.1 g of magnesium sulfate heptahydrate, 0.0001 g of manganese sulfate, 0.0001 g of ferric sulfate, 10.0 g of agar powder, 5.0 g of tricalcium phosphate, and 1000 mL of water), and Bacillus subtilis (strain 92068) was set as a positive control. They were cultured at room temperature, the colonies were washed off with water, and the diameters of phosphorus degradation halos were determined in mm.

inorganicphosphorus degrading capability=the diameter of the inorganicphosphorus degradation halo in mm+X

Note: X was a weighting coefficient, which was −2, −1, 0, 1 and 2 accordingly according to the transparency of the phosphorus degradation halo of the strain.

### Determination Results of Inorganicphosphorus Degrading Capability of KY240 Strain

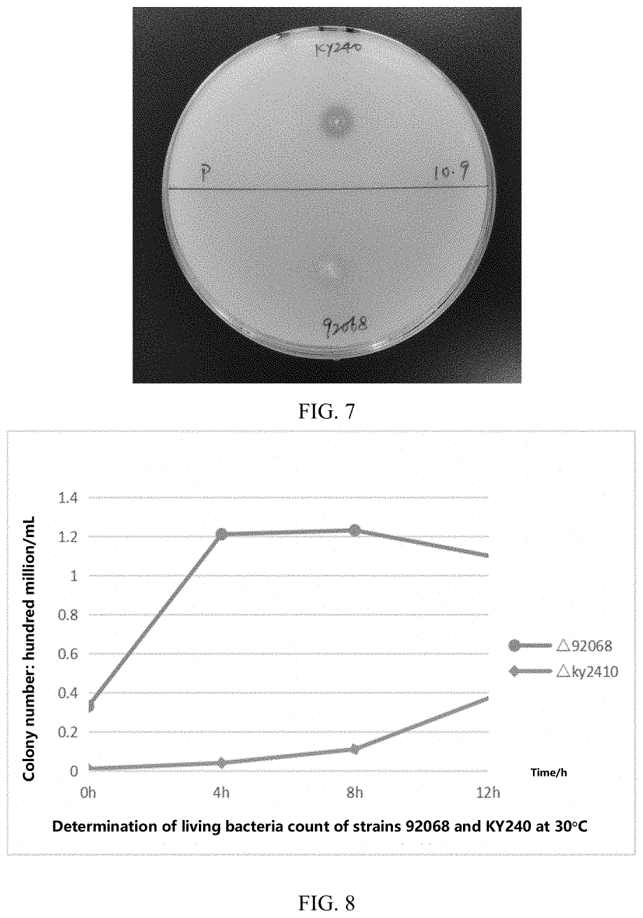

The results were as shown in Table 3 and FIG. 7. The results showed that the inorganicphosphorus degrading capability of the KY240 strain was obviously higher than that of the control Bacillus subtilis strain 92068.

TABLE 3
inorganicphosphorus degrading capability of strains on
an inorganicphosphorus medium

| | 92068 (CK) | KY240 |
|---|---|---|
| Strain | Bacillus subtilis | Pseudomonas graminis |
| Inorganicphosphorus | <5 | 27.2 |
| degrading | | |
| capability | | |

### Determination of Growth of KY240 Strain at 30° C./4° C.

The screened strains were inoculated into 50 mL of a R2A liquid medium (0.50 g of yeast powder, 0.50 g of peptone, 0.50 g of tryptone, 0.50 g of glucose, 0.50 g of soluble starch, 0.30 g of dipotassium hydrogen phosphate, 0.30 g of sodium pyruvate, 0.05 g of magnesium sulfate, and 1000 mL of water), and subjected to shake-flask culture at 30° C./4° C. and 200 rpm overnight. The R2A liquid medium was used as the positive control. OD_{600 nm} of the strain was determined, and the strain was re-inoculated in 100 mL of the R2A liquid medium at the quantified OD_{600 nm}=0.05, and the living bacteria counts of the strain at (30° C.: 4 h, 8 h, 12 h, 24 h)/(4° C.: 0 h, 24 h, 48 h, 72 h) was determined respectively. The bacterial solution at each time point was taken and diluted to a proper dilution, and then plated onto a petri dish. Each dilution was made in triplicates, and the petri dish was cultured in a 30° C. incubator to calculate the average number of colonies. A curve graph was depicted with the time/h as the abscissa and the effective colony number as the ordinate. The Bacillus subtilis strain 92068 was set as the positive control.

### Result

### Determination of Living Bacteria Count of KY240 Strain at 30° C.

The experimental results were as shown in FIG. 8. It could be seen from FIG. 8 that at 30° C., the number of the KY240 strain reached 38 million/mL at 12 h, and the strain 92068, which was a high-temperature growth strain, reached a peak of 120 million/mL at 8 h, which was 3 times that of the KY240 strain, and the KY240 strain could continue to grow after 12 h.

### Determination of Living Bacteria Count of KY240 Strain at 4° C.

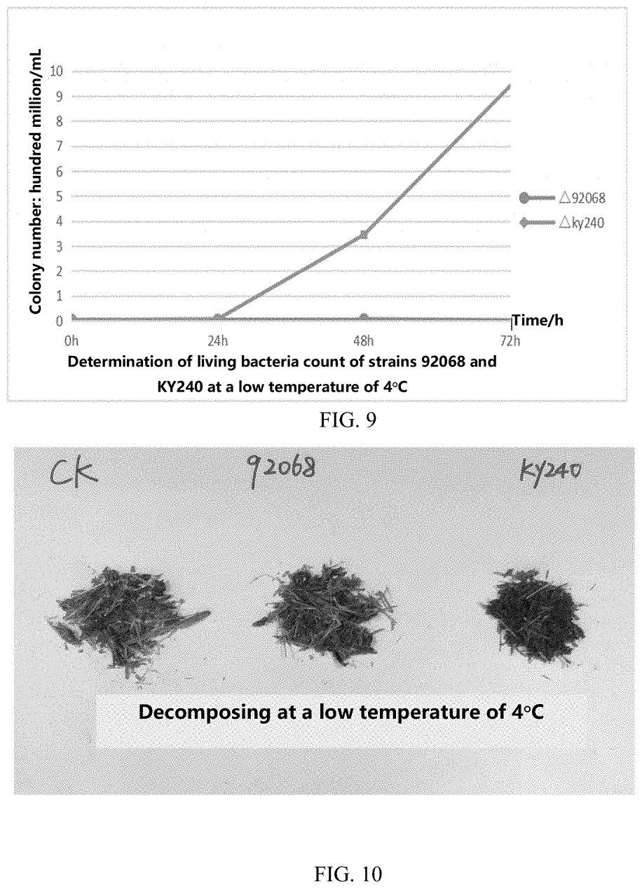

The experimental results were as shown in FIG. 9. It could be seen from FIG. 9 that the KY240 strain could grow rapidly and stably at 4° C. and reached 940 million/mL at 72 h, in contrast the strain 92068 did not grow at 72 h.

### Determination of Wheat Straw Decomposition Capability of KY240 Strain at the Condition of 4° C.

Wheat straws were taken as the substrate. 3 g of them were weighed into each test tube, and each test tube was added with a small and equal amount of liquid R2A to make it wet, added with 1 mL of the bacteria solution, shaken uniformly, cultured at a low temperature of 4° C. for decomposition, and timely and appropriately irrigated with equal amount of the bacteria solution according to its growth requirements, and the decomposition of the wheat straws were observed every week. The R2A liquid medium on which no bacteria was inoculated was set as the blank control CK1, and the R2A liquid medium inoculated with the Bacillus subtilis 92068 was set as the positive control CK2.

### Experimental Results of Wheat Straw Decomposition Capability of KY240 Strain at the Condition of 4° C.

The experimental results were as shown in FIG. 10. The results showed that at a low temperature (4° C.), the decomposed degree and speed of the wheat straws decomposed by the KY240 strain were significantly higher than those of the wheat straws decomposed by the Bacillus subtilis strain 92068.

Conclusion: the KY240 strain could degrade cellulose at a low temperature of 4° C., and upon sequencing analysis of the 16sDNA of the strain, the strain was highly homologous to Pseudomonas graminis. Compared with a commercial strain Bacillus subtilis 92068, the KY240 strain has stronger capability of degrading cellulose at a low temperature of 4° C. and a condition of 10° C., and the cellulose degradation capacities are basically the same. Although the cellulose degradation capacity is weakened at 30° C., the degradation amplitude is not large, and meanwhile, the KY240 strain also has potassium and inorganicphosphorus degradation capacity, which has a promotion effect on the decomposition of wheat straws, and the effect is obviously superior to that of the commercial strain 92068. Therefore, KY240 is a functional microbial strain with both a wide working range and multiple functions, which can both degrade cellulose at a low temperature (4° C.) and medium-high temperatures (10° C.-30° C.), and has the capability of degrading both potassium and inorganicphosphorus, and has a promotion effect on the decomposition of wheat straws. It is expected to be used for preparing bacterial fertilizer for degrading cellulose, promoting straw decomposition, and improving soil fertility, especially increasing the amount of potassium and phosphorus in soil available to plants.

What is claimed is:

1. A Pseudomonas graminis strain preserved in China General Microbiological Culture Collection Center (CGMCC) of Institute of Microbiology, Chinese Academy of Sciences, located at No. 3, No. 1 Courtyard, Beichen West Road, Chaoyang District, Beijing, with the accession number of CGMCC No.: 18751.

2. A microbial preparation comprising the Pseudomonas graminis strain according to claim 1.

3. The microbial preparation according to claim 2, wherein it is used in at least one of the following aspects:
degrading an organic matter containing cellulose;
promoting straw decomposition;
degrading potassium and inorganicphosphorus; and
preparing microbial fertilizer.

4. Use of the Pseudomonas graminis strain according to claim 1, comprising:
degrading an organic matter containing cellulose;
promoting straw decomposition;
degrading potassium and inorganicphosphorus; or
preparing microbial fertilizer.

5. A method for promoting cellulose degradation, comprising culturing a mixture of the Pseudomonas graminis strain according to claim 1 and cellulose.

6. The method according to claim 5, wherein the culture temperature is 0-40° C.

7. A method for degrading potassium, comprising culturing a mixture of the Pseudomonas graminis strain according to claim 1 and a potassium-containing mineral.

8. The method according to claim 7, wherein the potassium-containing mineral is selected from potassium feldspar, plagioclase, microcline, illite, vermiculite, montmorillonite, silicate and mica.

9. A method for degrading inorganicphosphorus, comprising culturing a mixture of the Pseudomonas graminis strain according to claim 1 and a phosphorus-containing mineral.

10. The method according to claim 9, wherein the phosphorus-containing mineral is selected from tricalcium phosphate, rock phosphate powder, fluorapatite, chloroapatite, hydroxyapatite, goyazite, vivianite, lignite, peat soil, wavellite, variscite and strengite.
